(19) United States
(12) Patent Application Publication
(10) Pub. No.: US 2025/0104861 A1
(43) Pub. Date: Mar. 27, 2025

(54) LARGE-SCALE TRAINING OF FOUNDATION MODELS FOR PHYSIOLOGICAL SIGNALS FROM WEARABLE ELECTRONIC DEVICES

(71) Applicant: Apple Inc., Cupertino, CA (US)

(72) Inventors: Hamidreza ABBASPOURAZAD, San Diego, CA (US); Udhyakumar NALLASAMY, San Jose, CA (US); Oussama ELACHQAR, Seattle, WA (US); Saba EMRANI, Belmont, CA (US); Andrew MILLER, Brooklyn, NY (US); Ian R. SHAPIRO, San Francisco, CA (US)

(21) Appl. No.: 18/665,282

(22) Filed: May 15, 2024

Related U.S. Application Data
(60) Provisional application No. 63/539,798, filed on Sep. 21, 2023.

(51) Int. Cl. G16H 50/20; G16H 10/60

(52) U.S. Cl. CPC G16H 50/20; G16H 10/60

(57) ABSTRACT

The subject technology provides for large-scale training of foundation models for physiological signals from wearable electronic devices. An apparatus receives receive input data having a plurality of physiological signal information segments associated with a user. The apparatus applies one or more augmentation functions to the plurality of physiological signal information segments to generate an augmented version of the plurality of physiological signal information segments. The apparatus trains a neural network to produce a trained machine learning model by generating, via an encoder, an embedding of the augmented version having a first number of dimensions in an embedding space. The apparatus maps, via a multilayer perceptron projection, the embedding into a representation having a second number of dimensions. The apparatus determines mutual information between a pair of representations of the augmented version. The apparatus can deploy the trained machine learning model to predict a physiological state of the user.

### CROSS-REFERENCE TO RELATED APPLICATION(S)

This application claims the benefit of U.S. Provisional Application Ser. No. 63/539,798, entitled “LARGE-SCALE TRAINING OF FOUNDATION MODELS FOR PHYSIOLOGICAL SIGNALS FROM WEARABLE ELECTRONIC DEVICES,” and filed on Sep. 21, 2023, the disclosure of which is expressly incorporated by reference herein in its entirety.

### TECHNICAL FIELD

The present description generally relates to large-scale training of foundation models for physiological signals from wearable electronic devices.

### BACKGROUND

Various physiological parameters of a user can be measured and analyzed to estimate other physiological measures indicative of the user's physiological state. Computer hardware has been utilized to make improvements across different industry applications including applications used to assess and monitor physiological activities.

### BRIEF DESCRIPTION OF THE DRAWINGS

Certain features of the subject technology are set forth in the appended claims. However, for purpose of explanation, several embodiments of the subject technology are set forth in the following figures.

FIG. 1 illustrates an example network environment in accordance with one or more implementations.

FIG. 2 illustrates an example computing architecture for a system providing for large-scale training of foundation models in accordance with one or more implementations.

FIG. 3 is a flow chart of an example process that may be performed for large-scale training of foundation models in accordance with one or more implementations.

FIG. 4 is a schematic diagram of an example pre-training framework in accordance with one or more implementations.

FIG. 5A illustrates an example encoder architecture in accordance with one or more implementations.

FIG. 5B illustrates an example architecture of a mobile inverted bottleneck convolutional block in accordance with one or more implementations.

FIG. 6 conceptually illustrates an example overview of comparison of predictions between different types of embeddings using large-scale training of foundation models in accordance with one or more implementations.

FIG. 7 illustrates an electronic system with which one or more implementations of the subject technology may be implemented.

### DETAILED DESCRIPTION

The detailed description set forth below is intended as a description of various configurations of the subject technology and is not intended to represent the only configurations in which the subject technology can be practiced. The appended drawings are incorporated herein and constitute a part of the detailed description. The detailed description includes specific details for the purpose of providing a thorough understanding of the subject technology. However, the subject technology is not limited to the specific details set forth herein and can be practiced using one or more other implementations. In one or more implementations, structures and components are shown in block diagram form in order to avoid obscuring the concepts of the subject technology.

Machine learning has seen a significant rise in popularity in recent years due to the availability of training data, and advances in more powerful and efficient computing hardware. Machine learning may utilize models that are executed to provide predictions in particular applications.

Recent advancements in wearable device technology have led to the capability to record various physiological signals, which can be utilized for monitoring the overall wellness of users. Two of the most commonly collected physiological signals from wearable devices are photoplethysmography (PPG) and electrocardiogram (ECG). Cardiac electrical activity is measured by ECG, containing information about cardiovascular health, while volumetric changes in arterial blood flow are measured by PPG, encompassing a wide range of biological information. These physiological signals are employed on a wearable electronic device for the detection of certain health conditions, such as atrial fibrillation, and the monitoring of specific health metrics, such as blood oxygen. Despite the significant potential of these physiological signals, the development of digital-based physiological markers using deep neural networks has been hindered by the absence of curated datasets with annotated medical labels. Lengthy and costly health studies typically collect medical datasets, necessitating domain expertise for annotation and often involving only a few users, potentially limiting the generalizability of learned models to the population demographics.

Inspired by advancements in foundation models for language and vision, the use of large-scale pretraining for physiological signals is explored in the present disclosure. Large-scale training of foundation models via self-supervised learning (SSL) has shown promise in domains such as natural language processing, computer vision, and speech recognition, and has recently been applied in health applications using physiological signals recorded in hospital or controlled experimental settings. Self-supervised learning often does not necessitate explicit labels, making it suitable for the pre-training of foundation models on unlabeled physiological signals.

Physiological signal foundation models with PPG and ECG can be trained using a large-scale data collection process. In large-scale pre-training for physiological signals, the models for physiological signals are trained on a large-scale dataset collected via wearable electronic devices with approximately 150,000 users. In self-supervised training framework, techniques from self-supervised learning for physiological signals are combined with methods from other domains, such as computer vision. The self-supervised pre-training incorporates a stochastic user-level augmentation module, and the encoder is optimized using momentum training with a regularized normalized cross-entropy (NCE) loss (or a modified version of InfoMax loss used in self-supervised learning tasks). Examination of the amount of information encoded by the pre-trained foundation models across various targets demonstrates that pre-trained PPG/ECG embeddings contain predictive information concerning a range of demographic variables and health conditions. The investigation encompasses the assessment of information encoded in the PPG/ECG embeddings of pre-trained models with respect to various targets, including demographics and health conditions.

The subject technology provides techniques for large-scale training of foundation models for physiological signals from wearable electronic devices. A method includes receiving input data comprising physiological signal information associated with a user; generating a latent representation of the input data comprising an informative structure of the physiological signal information; and producing a trained machine learning model by training a neural network with the latent representation using one or more self-supervised learning tasks to predict a physiological state of the user.

Implementations of the subject technology improve the ability of a given electronic device to provide sensor-based, machine-learning generated feedback to a user (e.g., a user of the given electronic device). These benefits therefore are understood as improving the computing functionality of a given electronic device, such as an end user device which may generally have less computational and/or power resources available than, e.g., one or more cloud-based servers. Additionally, several advantages are associated with the development of foundation models for physiological signals, including the following: 1) a reduced amount of labeled data is required to achieve the same level of accuracy as supervised models, resulting in significant cost reductions for experimental health studies, 2) these models can be further fine-tuned for downstream targets, leading to improved accuracy and requiring fewer computational resources due to faster convergence compared to training from scratch, and 3) they yield signal-to-embedding models that can be utilized to calculate similarity scores between users or signals, facilitating faster information retrieval.

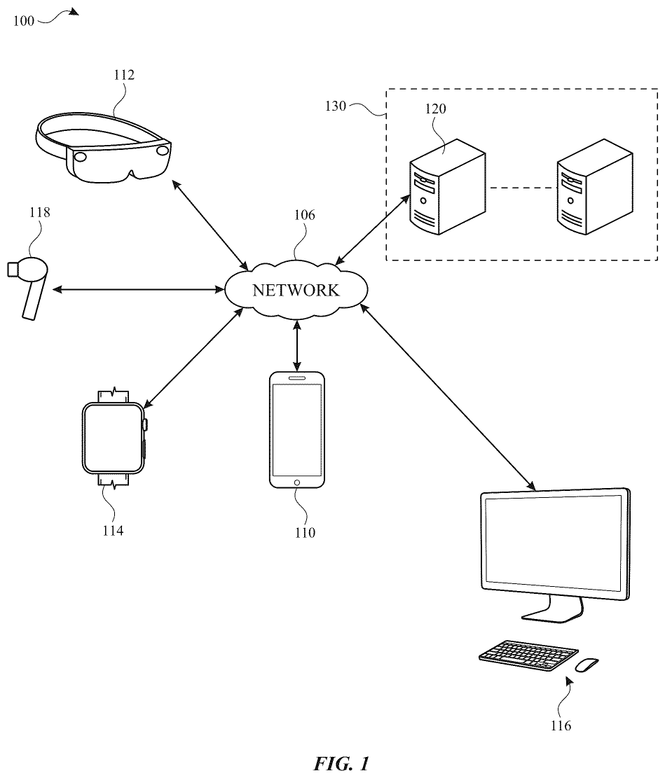

FIG. 1 illustrates an example network environment 100 in accordance with one or more implementations. Not all of the depicted components may be used in all implementations, however, and one or more implementations may include additional or different components than those shown in the figure. Variations in the arrangement and type of the components may be made without departing from the spirit or scope of the claims as set forth herein. Additional components, different components, or fewer components may be provided.

The network environment 100 includes an electronic device 110, an electronic device 112, an electronic device 114, an electronic device 116, an electronic device 118, and a server 120. The network 106 may communicatively (directly or indirectly) couple the electronic device 110 and/or the server 120. In one or more implementations, the network 106 may be an interconnected network of devices that may include, or may be communicatively coupled to, the Internet. For explanatory purposes, the network environment 100 is illustrated in FIG. 1 as including the electronic device 110, the electronic device 112, the electronic device 114, the electronic device 116, the electronic device 118, and the server 120; however, the network environment 100 may include any number of electronic devices and any number of servers or a data center including multiple servers.

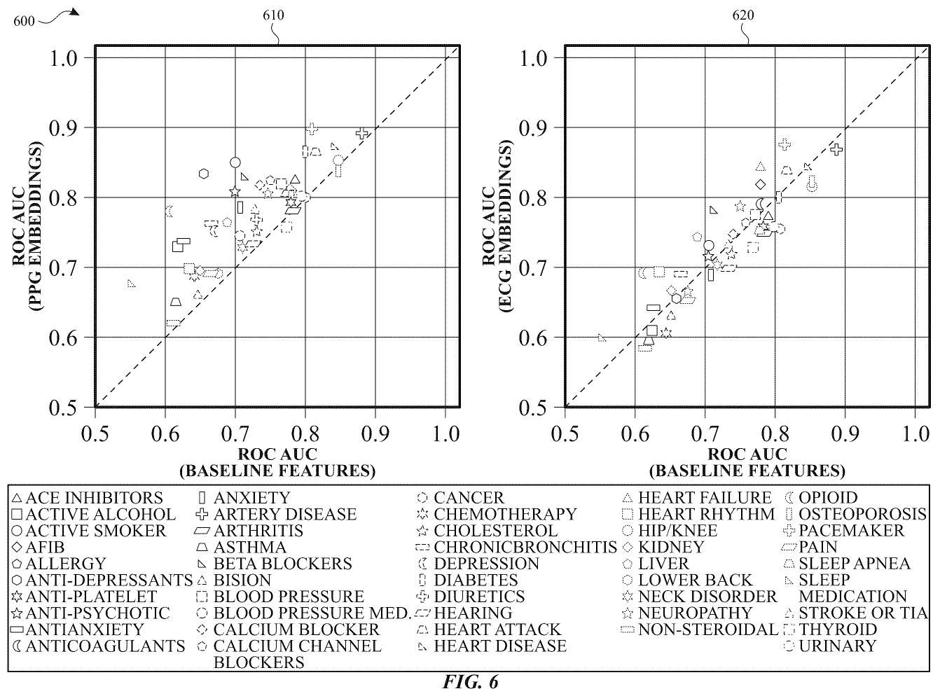

The electronic device 110 may be, for example, a desktop computer, a portable computing device such as a laptop computer, a smartphone, a peripheral device (e.g., a digital camera, headphones), a tablet device, a wearable device such as a watch, a band, and the like. In FIG. 1, by way of example, the electronic device 110 is depicted as a mobile electronic device (e.g., smartphone). The electronic device 110 may be, and/or may include all or part of, the electronic system discussed below with respect to FIG. 6.

The electronic device 112 may be, for example, desktop computer, a portable computing device such as a laptop computer, a smartphone, a peripheral device (e.g., a digital camera, headphones), a tablet device, or a wearable device such as a head mountable portable system, that includes a display system capable of presenting a visualization of an extended reality environment to a user. In FIG. 1, by way of example, the electronic device 112 is depicted as a head mountable portable system. The electronic device 112 may be, and/or may include all or part of, the electronic system discussed below with respect to FIG. 6.

The electronic device 114 may be, for example, desktop computer, a portable computing device such as a laptop computer, a smartphone, a peripheral device (e.g., a digital camera, headphones), a tablet device, a wearable device such as a watch, a band, and the like. In FIG. 1, by way of example, the electronic device 114 is depicted as a watch. The electronic device 114 may be, and/or may include all or part of, the electronic system discussed below with respect to FIG. 6.

The electronic device 116 may be, for example, desktop computer, a portable computing device such as a laptop computer, a smartphone, a peripheral device (e.g., a digital camera, headphones), a tablet device, a wearable device such as a watch, a band, and the like. In FIG. 1, by way of example, the electronic device 116 is depicted as a desktop computer. The electronic device 116 may be, and/or may include all or part of, the electronic system discussed below with respect to FIG. 6.

The electronic device 118 may be, for example, desktop computer, a portable computing device such as a laptop computer, a smartphone, a peripheral device (e.g., a digital camera, headphones), a tablet device, a wearable device such as a watch, a band, and the like. In FIG. 1, by way of example, the electronic device 118 is depicted as a earbud. The electronic device 118 may be, and/or may include all or part of, the electronic system discussed below with respect to FIG. 6.

In one or more implementations, one or more of the electronic devices 110-118 may provide a system for training a machine learning model using training data, where the trained machine learning model is subsequently deployed to one or more of the electronic devices 110-118. Further, one or more of the electronic devices 110-118 may provide one or more machine learning frameworks for training machine learning models and/or developing applications using such machine learning models. In an example, such machine learning frameworks can provide various machine learning algorithms and models for different problem domains in machine learning. In an example, the electronic device 110 may include a deployed machine learning model that provides an output of data corresponding to a prediction or some other type of machine learning output. In one or more implementations, training and inference operations that involve individually identifiable information of a user of one or more of the electronic devices 110-118 may be performed entirely on the electronic devices 110-118, to prevent exposure of individually identifiable data to devices and/or systems that are not authorized by the user.

The server 120 may form all or part of a network of computers or a group of servers 130, such as in a cloud computing or data center implementation. For example, the server 120 stores data and software, and includes specific hardware (e.g., processors, graphics processors and other specialized or custom processors) for rendering and generating content such as graphics, images, video, audio and multi-media files. In an implementation, the server 120 may function as a cloud storage server that stores any of the aforementioned content generated by the above-discussed devices and/or the server 120.

The server 120 may provide a system for training a machine learning model using training data, where the trained machine learning model is subsequently deployed to the server 120 and/or to one or more of the electronic devices 110-118. In an implementation, the server 120 may train a given machine learning model for deployment to a client electronic device (e.g., the electronic device 110, the electronic device 112, the electronic device 114, the electronic device 118). In one or more implementations, the server 120 may train portions of the machine learning model that are trained using (e.g., anonymized) training data from a population of users, and one or more of the electronic devices 110-118 may train portions of the machine learning model that are trained using individual training data from the user of the electronic devices 110-118. The machine learning model deployed on the server 120 and/or one or more of the electronic devices 110-118 can then perform one or more machine learning algorithms. In an implementation, the server 120 provides a cloud service that utilizes the trained machine learning model and/or continually learns over time.

In the example of FIG. 1, the electronic device 110 is depicted as a smartphone. However, it is appreciated that the electronic device 110 may be implemented as another type of device, such as a wearable device (e.g., a smart watch or other wearable device). The electronic device 110 may be a device of a user (e.g., the electronic device 110 may be associated with and/or logged into a user account for the user at a server). Although a single electronic device 110 is shown in FIG. 1, it is appreciated that the network environment 100 may include more than one electronic device, including more than one electronic device of a user and/or one or more other electronic devices of one or more other users.

In one or more implementations, the physiological signals may include electromyography data recorded by at least one of the electronic devices 110-118, such as the electronic device 114, electroencephalography data recorded by at least one of the electronic devices 110-118, such as the electronic device 118, electrocardiography data recorded by at least one of the electronic devices 110-118, such as the electronic device 114, electrooculography data recorded by at least one of the electronic devices 110-118, such as the electronic device 118, and respiration data recorded by at least one of the electronic devices 110-118, such as the electronic device 118, among others.

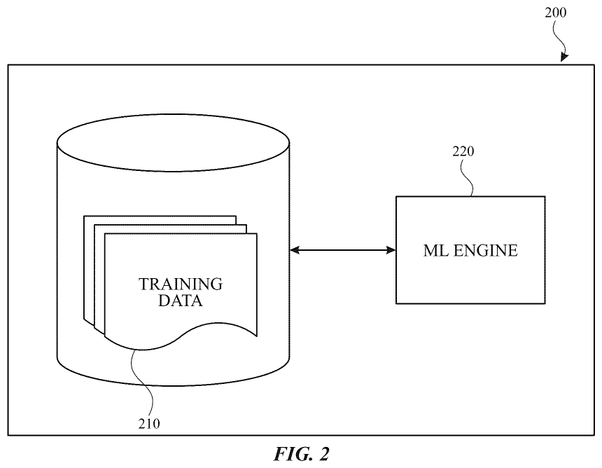

FIG. 2 illustrates an example computing architecture for a system providing for large-scale training of foundation models in accordance with one or more implementations. For explanatory purposes, the computing architecture is described as being provided by an electronic device 200, such as by a processor and/or memory of the server 120, or by a processor and/or a memory of any other electronic device, such as the electronic device 110. Not all of the depicted components may be used in all implementations, however, and one or more implementations may include additional or different components than those shown in the figure. Variations in the arrangement and type of the components may be made without departing from the spirit or scope of the claims as set forth herein. Additional components, different components, or fewer components may be provided.

Machine learning model 220 may include one or more neural networks. The system can perform model selection by selecting a suitable machine learning algorithm, such as decision trees, neural networks, or support vector machines, that can learn from the pre-processed data and perform the desired actions, such as predicting a physiological state of a user.

In one or more implementations, machine learning model 220 includes one or more convolutional neural networks that may be characterized by a compound scaling method that optimizes both the depth and width of the neural network. In one or more implementations, machine learning model 220 employs an encoder, a projection head, 16 mobile-inverted bottleneck blocks and a 256-dimensional embedding (the representation vector after the encoder). The encoder may consist of 3.3 million parameters for PPG and 2.5 million for ECG physiological signals. The projection head may include a multi-layer perceptron with one hidden layer that includes 1024 units, which can transform the 256-dimensional embedding into a 128-dimensional representation subspace where the loss can be calculated.

In one or more implementations, the system employs NCE to maximize the mutual information between the representations of positive pairs, while also permitting contrast with other positive pairs within each batch through the utilization of the cross-entropy function. This is done to prevent representation collapse. Additionally, to promote a uniform distribution of features within the batch, the Kozachenko-Leonenko (KoLeo) differential entropy is used as a form of regularization. In one or more implementations, a temperature value in the range of 0 to 1 (e.g., 0.04) can be utilized for both PPG and ECG modalities in the case of NCE, and the weight for KoLeo regularization in the objective function can be set at a weight value in the range of 0 to 1 (e.g., 0.1). For bootstrap-your-own-latent (BYOL), the prediction head may feature a multi-layer perceptron with one hidden layer containing 1024 units. In some aspects, batch normalization can be employed to prevent collapse in BYOL. In this regard, machine learning model 220 may include batch normalization in the projection and prediction (if applicable) heads.

The system can perform training of the machine learning model by training the selected model on the pre-processed data. This involves splitting the data into training, validation, and test sets, setting hyperparameters, and using an optimization algorithm to minimize the model's loss or error on the training data. In an example, the electronic device 110 and/or the server 120 may utilize one or more machine learning algorithms that uses training data 210 for training the ML model 220.

In one or more implementations, the training of machine learning model 220 may employ a batch size of 256, with an initial learning rate of 0.001 for non-contrastive methods and 0.00025 for contrastive methods. The training of machine learning model 220 may utilize step learning rate scheduling to expedite convergence. The machine learning model 220 may undergo training for a maximum of 100 million iterations, in one or more implementations. The PPG and ECG segments can be pre-processed, and temporal channel-wise z-scoring can be performed on each segment.

As illustrated, the electronic device 200 includes training data 210 for training a machine learning model. The system can perform data pre-processing by pre-processing the collected data to make it suitable for training the machine learning model. This includes data cleaning, normalization, feature extraction, and feature engineering. Training data 210 may include activity information associated with physiological activity. For example, the activity information may include user activity measurements associated with user interactions and/or other user engagement activities based on time and/or location. In one or more implementations, training data 210 may include training data obtained by a device on which a trained machine learning (ML) model 220 is deployed and/or training data obtained by other devices.

Training data 210 may include activity information associated with measurable physiological signals or electrical impulses generated within a user. These signals are produced by various physiological processes in the body and carry important information about the user's health, function, and state. These physiological signals can be broadly categorized into different types, including: (1) Electrocardiogram (ECG/EKG), (2) Electroencephalogram (EEG); (3) Electromyogram (EMG); (4) Electrooculogram (EOG); (5) Electrodermal Activity (EDA); (6) Electroencephalography (ECOG); (7) Electroretinogram (ERG); (8) Electrogastrogram (EGG); and (9) Electrocorticography (ECoG). In some aspects, ECG/EKG may refer to a physiological signal that measures the electrical activity of the heart. It is commonly used to assess heart rate, rhythm, and detect abnormalities in the heart's function. In some aspects, EEG measures the electrical activity of the brain. EEG can be used for potentially identifying and studying neurological conditions, monitoring brain activity during sleep, and understanding cognitive processes. In some aspects, EMG may refer to a physiological signal measures the electrical activity of muscles. It is useful in potentially identifying neuromuscular disorders, assessing muscle function, and monitoring physical rehabilitation progress. In some aspects, EOG records the electrical activity of the muscles around the eyes and is commonly used to monitor eye movements and detect abnormalities related to vision and sleep. In some aspects, EDA may refer to a galvanic skin response (GSR) that measures the electrical conductance of the skin, which can provide information about emotional responses, stress, and arousal. In some aspects, ECOG may be similar to EEG but involves placing electrodes directly on the brain's surface, often used in neurosurgery or research. In some aspects, ERG measures the electrical responses of the retina in the eye, aiding in the potential identification of various visual disorders. In some aspects, EGG records the electrical activity of the stomach and can help in understanding gastric motility and digestive disorders. In some aspects, ECoG involves placing electrodes directly on the brain's surface, used for research and certain clinical applications like epilepsy monitoring. Training data 210 may also include demographic information (e.g., age, gender, body mass index (BMI), etc.) for a user of the electronic device 110, and/or a population of other users.

The system can perform data collection by collecting and curating a large dataset that contains examples of physiological signals. The effectiveness of self-supervised learning in health applications can be demonstrated, typically with small datasets comprising a few hundred or a few thousand users. For example, the training data 210 can encompass 12-lead ECG data in hospital settings or EEG data collected in controlled experimental settings.

In one or more implementations, training data 210 includes a separate pre-training dataset for PPG and ECG physiological signals. For a PPG-based pre-training dataset, approximately 20 million PPG segments can be curated from a general population of users (e.g., approximately 150,000 users). Random selection of 20 million PPG segments can be performed based on two conditions: 1) ensuring that each user can contribute at least 4 segments to the pre-training dataset, and 2) striving for uniformity in the number of segments per user. These PPG segments can have a duration of 60 seconds, sampled at a frequency of 64 Hz or 256 Hz, and can be characterized by four separate optical channels corresponding to different spatial combinations of transmitting and receiving diodes. PPG segments can be pre-processed using dark subtraction (to reject signals introduced by ambient light), followed by bandpass filtering, down-sampling to 64 Hz if needed and temporal channel-wise z-scoring for each PPG segment. For an ECG pre-training dataset, approximately 3.75 million ECG segments can be curated from approximately 106,000 users, applying the same two conditions as for the PPG pre-training dataset. These ECG segments can have a duration of 30 seconds, initially sampled at a frequency of 512 Hz, and subsequently down-sampled to 128 Hz. Unlike the PPG segments, the ECG segments can feature a single channel.

Training data 210 may also include demographic information (e.g., age, gender, personal preferences, etc.) for a user of the electronic device 110, and/or a population of other users. The activity information may also include locations (e.g., an indoor location, an outdoor location, a geographical location such as a Global Positioning System (GPS) location, or other location information) of one or more portions of an activity and/or weather conditions at the time of an activity. The activity information may have been obtained over the course of multiple (e.g., many) prior activities by a user of the electronic device 110, and/or by a population of other users, such as users that were wearing wearable devices during prior activities, and authorized collection of anonymized activity collections from the wearable devices. In one or more implementations, activity collections included in the training data 210 may include places visited, contacts and/or other people visited, a routine activity, a workout length in time or in distance, health measurements, and/or the like.

Recently, self-supervised learning has garnered popularity in various domains of deep learning. In some aspects, the self-supervised learning technique can employ joint embedding architectures (JEAs). In JEAs, training typically involves the utilization of a carefully designed loss function aimed at preventing representation collapse. Contrastive losses, such as NCE, prevent collapse by contrasting various samples within the batch, while non-contrastive losses prevent collapse through the application of momentum training or some form of regularization, or a combination of both. Self-supervised pre-trained models have been demonstrated to encode a significant amount of information about downstream targets, even in the absence of any labeled data during the pre-training phase.

The subject technology provides for pre-training foundation models for wearable PPG and ECG physiological signals by leveraging self-supervised contrastive learning to pre-train a deep neural network, such as an encoder. The machine learning model 220 can be trained and implemented to perform actions of a system for providing predictions of a physiological state of a user.

In one or more implementations, the system employs a user-level positive pair selection strategy for pre-training of the machine learning model 220, which contributes significantly to the enhancement of the quality and quantity of information contained in the learned embeddings. To encourage the extraction of user-level information by the model, the positive pairs are chosen from augmented views of two distinct segments originating from the same user. The notation for segment i belonging to user s is denoted as xᵢ⁸; hence, positive pairs are randomly selected in the form of {xᵢ⁸}, xⱼ⁸)|i≠j}.

In one or more implementations, the system employs an augmentation module T (⋅) that encompasses a stochastic collection of time-series augmentations. In some aspects, the system also employs a standard set of time-series augmentation functions, such as crop, add Gaussian noise, time warp, magnitude warp, and channel swap. Each augmentation function within the augmentation set is associated with a configurable probability value, and during each invocation of T (⋅), a sequence of augmentation functions from the augmentation set is applied based on randomly generated binary events determined by the assigned probability values. Additionally, each augmentation incorporates internally selected hyperparameters that are not subject to tuning in this study and fall outside its scope. Due to the various sources of randomness within the augmentation module, it introduces a wide range of distortions to the input segment. The intensity of these distortions is controlled by adjusting the probability values. For PPG, in one or more implementations, the augmentation set and corresponding probability values can be as follows: {cut out: 0.4, magnitude warp: 0.25, add Gaussian noise: 0.25, channel permute: 0.25, time warp: 0.15}, and for ECG: {cut out: 0.8, magnitude warp: 0.5, add Gaussian noise: 0.5, time warp: 0.3}.

In one or more implementations, the system employs NCE to maximize the mutual information between the representations of positive pairs, while also permitting contrast with other positive pairs within each batch through the utilization of the cross-entropy function. This is done to prevent representation collapse. Additionally, to promote a uniform distribution of features within the batch, the Kozachenko-Leonenko (KoLeo) differential entropy is used as a form of regularization.

The objective of the model for each batch of embeddings h derived from N positive pairs (h1, h2) is defined as follows:

$${L}_{contrastive}^{( 1 , 2 )} = - \frac{1}{N} {\sum }_{i = 1}^{N} log \frac{exp ( sim ( {h}_{1}^{i} , {h}_{2}^{i} ) / τ )}{{\sum }_{j = 1}^{N} 1 [ j \neq 1 ] exp ( sim ( {h}_{1}^{i} , {h}_{2}^{j} ) / τ )} , \qquad ( 1 )$$

where sim (·,·) is the cosine similarity function. KoLeo regularization is also calculated as:

$${L}_{KoLeo}^{( 1 )} = - \frac{1}{N} {\sum }_{i = 1}^{N} log ( {min}_{j \neq 1} {‖ {h}_{1}^{i} - {h}_{1}^{j} ‖}^{2} ) . \qquad ( 2 )$$

The final objective is computed as the regularized NCE loss:

$$L = \frac{1}{2} ( {L}_{contrastive}^{( 1 , 2 )} + {L}_{contrastive}^{( 2 , 1 )} ) + \frac{λ}{2} ( {L}_{KoLeo}^{( 1 )} + {L}_{KoLeo}^{( 2 )} ) . \qquad ( 3 )$$

In one or more implementations, both of the contrastive and regularization losses are calculated after L2-normalization of the embeddings.

While the necessity of utilizing momentum training to prevent representation collapse is optional, the system employs momentum training to introduce greater dissimilarity between the representations of positive pairs. This, in turn, further encourages the neural network to acquire informative representations rather than trivial ones. This approach is particularly advantageous for physiological signals such as ECG that exhibit reduced variability within users. Momentum training is applied to both the encoder and projection head, with the online side of the joint embedding architecture (JEA) being updated through backpropagation and the momentum side being maintained as a trailing exponential moving average of the online side. The momentum update rule can be as follows: ξ←τξ+(1−τ)θ, where θ represents the weights of the online side, ξ represents the weights of the momentum side, and τ denotes the momentum update rate. In one or more implementations, a constant momentum update rate of 0.99 can be utilized.

The system can perform model evaluation by evaluating the trained model on the validation and test sets to ensure that it performs well and generalizes to new data. This includes calculating metrics such as accuracy, precision, recall, and F1-score.

In one or more implementations, the system may conduct linear probing for the prediction of users' age, BMI, and biological gender. In the case of classification tasks, ridge regression can be performed to predict scores for binarized targets (0/1). The performance can be assessed using metrics such as the area under the curve (AUC) of a receiver operating characteristic (ROC) and partial AUC (pAUC) at a false positive rate (FPR) of about 10%. For regression tasks, ridge regression can be performed to predict continuous targets and the performance can be quantified using mean absolute error (MAE). For age prediction, the system can perform linear classification to distinguish ages above 50 from ages below 50, and the system can further carry out linear regression to estimate age values. Regarding BMI, the system can employ linear classification to differentiate between BMI values above 30 and those below 30, and the system can utilize linear regression to predict BMI values. For biological gender, the system can conduct linear classification to distinguish between biological males and biological females.

In one or more implementations, the system may execute linear probing at a user level, where all the embeddings associated with each user are mean-aggregated, resulting in each user contributing only one sample for downstream training and evaluation. Furthermore, the splits for downstream training and evaluation can be stratified based on users, ensuring that there is no overlap between users in the evaluation split and those in the training split.

In one or more implementations, the system can receive responses from users based on a questionnaire covering various aspects of historical health records, demographics, and lifestyle habits associated with the users. These responses may typically be in the form of “yes” or “no,” indicating whether the user has had a history of specific health conditions (e.g., atrial fibrillation), has consumed particular medication (e.g., anti-depressants), or has had specific lifestyle habits (e.g., smoking). These questionnaire prompts can be mapped to various binary labels and can be predicted using binary classification. To establish a baseline, the predictive capabilities of the pre-trained encoder embeddings can be compared with baseline features, including age, biological gender, BMI, ethnicity, as well as average heart rate and standard deviation of heart rate derived from PPG physiological signals, which are known to provide informative insights into certain conditions. In one or more implementations, the system can evaluate the linear classification for these targets using AUC based on PPG embeddings and compared with baseline features.

The concept of a smooth effective rank, an unsupervised metric that quantifies the entropy of a singular value distribution of embeddings within a given batch, can be utilized by the system as part of the evaluation. This metric can serve as a rough proxy for downstream evaluations without requiring any labels, as it is expected to exhibit a positive correlation with downstream evaluations. In one or more implementations, the smooth effective rank can be computed as the average value for batches of size 256 in the pre-training validation split.

The system can perform model deployment once the trained model has been evaluated and validated. The model can be deployed on a target application to perform the desired actions. This involves integrating the model into a target application's codebase and providing a user interface for users to interact with the model's outputs. Overall, training and implementing a machine learning model to perform actions in a target application may include a combination of data collection, pre-processing, model selection, training, evaluation, and deployment. It also may entail careful consideration of ethical and privacy concerns related to collecting and processing user data.

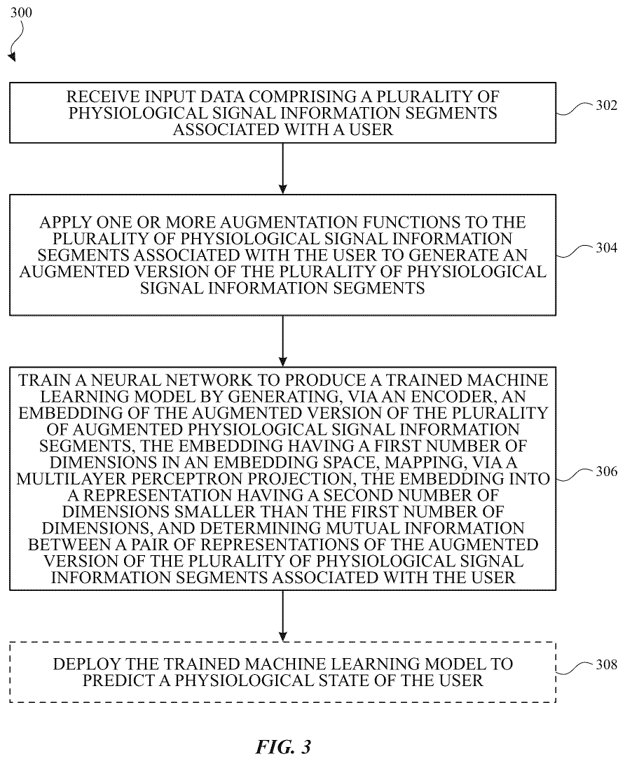

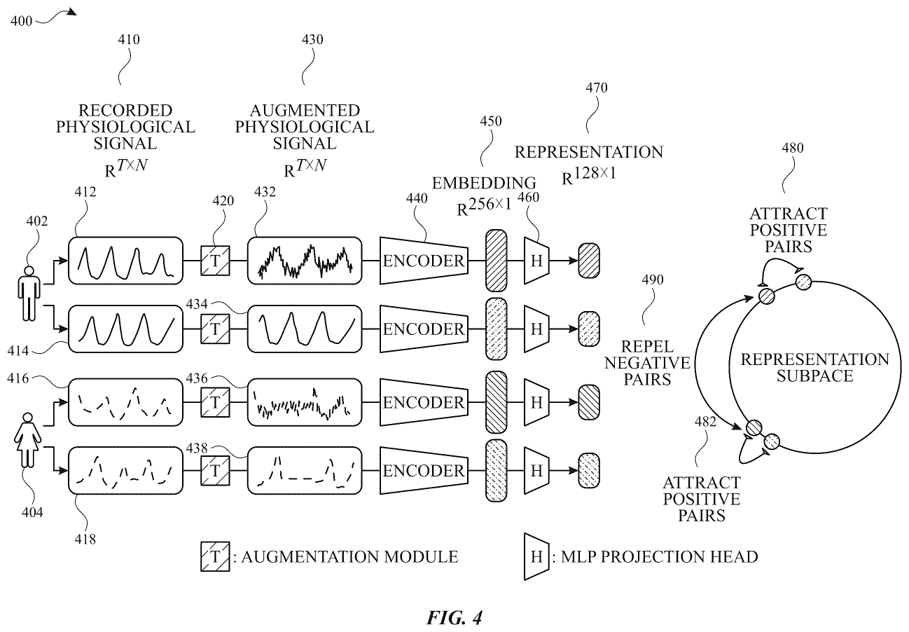

FIG. 3 is a flow chart of an example process 300 that may be performed for large-scale training of foundation models in accordance with one or more implementations. For explanatory purposes, the process 300 is primarily described herein with reference to the electronic device 110 of FIG. 1. However, the process 300 is not limited to the electronic device 110 of FIG. 1, and one or more blocks (or operations) of the process 300 may be performed by one or more other components of other suitable devices and/or servers. Further for explanatory purposes, some of the blocks of the process 300 are described herein as occurring in serial, or linearly. However, multiple blocks of the process 300 may occur in parallel. In addition, the blocks of the process 300 need not be performed in the order shown and/or one or more blocks of the process 300 need not be performed and/or can be replaced by other operations. In one or more implementations, the operations of the process 300 will be discussed with reference to FIG. 4 for purposes of explanation and brevity of discussion. FIG. 4 is a schematic diagram of an example pre-training framework 400 in accordance with one or more implementations.

As illustrated in FIG. 3, at block 302, an apparatus (e.g., the electronic device 110, 112, 114, 116, 118) receives input data comprising a plurality of physiological signal information segments associated with a user. The pre-training framework 400 provides a high-level visualization of a mini-batch involving 2 participants (e.g., 402, 404) and 4 segments (e.g., 412-418).

At block 304, the apparatus applies one or more augmentation functions to the plurality of physiological signal information segments associated with the user to generate an augmented version of the plurality of physiological signal information segments.

In one or more implementations, the process of the pre-training framework 400 involves passing recorded physiological signals 410 through an augmentation layer 420. As illustrated in FIG. 4, the recorded physiological signals 410 include segments 412 and 414 of participant 402 that are processed through the augmentation layer 420 to produce segments 432 and 434 of augmented physiological signals 430. For participant 404, the segments 416 and 418 of recorded physiological signals 410 are processed through the augmentation layer 420 to produce segments 436 and 438 of augmented physiological signals 430.

At block 306, the apparatus trains a neural network to produce a trained machine learning model by generating, via an encoder, an embedding of the augmented version of the plurality of augmented physiological signal information segments, the embedding having a first number of dimensions in an embedding space, mapping, via a multilayer perceptron projection, the embedding into a representation having a second number of dimensions smaller than the first number of dimensions, and determining mutual information between a pair of representations of the augmented version of the plurality of physiological signal information segments associated with the user.

Referring to FIG. 4, the augmented views of the physiological signals (e.g., the augmented physiological signals 430) are passed through an encoder 440 to produce embeddings 450, followed by a Multilayer Perceptron (MLP) projection head 460 to generate representations 470. These representations 470 are used in computing a contrastive loss function, which serves to draw positive pairs (e.g., 480, 482) closer together while simultaneously pushing negative pairs apart (e.g., 490). Positive pairs are defined as distinct segments originating from the same participant (e.g., 480, 482).

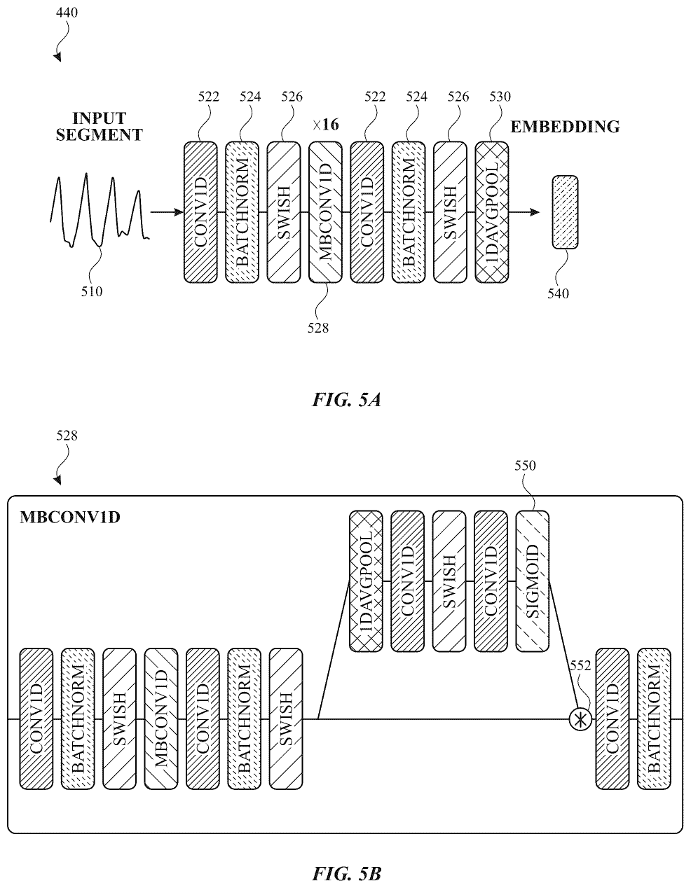

In one or more implementations, the encoder 440 is a one-dimensional convolutional neural network, incorporating 16 mobile-inverted bottleneck convolutional blocks 528 (see FIG. 5) with squeeze-and-excitation mechanisms. The MLP projection head 460, responsible for further dimensionality reduction of the 256-dimensional embedding 450, can be structured as a multi-layer perceptron with a single hidden layer containing 1024 units. A 256-dimensional embedding 450 (e.g., [symbol] ^{256×1}) can be employed consistently across all models, representing the output of the deep encoder. This reduction results in a 128-dimensional representation 470 subspace (e.g., [symbol] ^{128×1}) where the loss calculation occurs.

In one or more other implementations, the encoder 440 may incorporate batch normalization in its projection and prediction heads for consistency. A constant momentum update rate of 0.99 is applied across the pre-training framework 400. The batch size for model training is set at 256, with an initial learning rate of 0.001 in one or more implementations or 0.00025 in one or more other implementations. The pre-training framework 400 may employ a step learning rate scheduling to expedite convergence. In one or more other implementations, the augmentation layer 420 may be assigned with a higher probability for certain modalities to effective augmentations such as “cut out.” For example, higher probabilities may be assigned to ECG segments for ECG augmentation to allow for increased mismatch between positive pair representations.

In a contrastive loss function (e.g., normalized cross-entropy (NCE)) method, a temperature value of 0.04 is utilized for both PPG and ECG modalities. Additionally, a weight in the range of 0 to 1 (e.g., 0.1) can be assigned to the KoLeo regularization term within the objective function. The training process involves the use of the Adam optimizer, with gradient descent updates distributed across 32 GPUs, enabling efficient parallel processing. This comprehensive setup ensures effective training and optimization of the models for the specified tasks.

Pre-training foundation models using unlabeled data have been completed in various domains of deep learning such as natural language processing and computer vision. The foundation models for PPG and ECG modalities are pre-trained using self-supervised learning and a large longitudinal dataset. The pretrained foundation models can encode participant demographics with high accuracy and encode information predictive of a broad range of self-reported health conditions and medication categories. The target labels used for training and evaluating downstream classifiers can be constructed from participants' self-reported surveys. Although PPG/ECG embeddings can be predictive of health conditions, other physiological markers such as heart rate, heart rate variability (HRV), and physical activity offer valuable insight into one's health status. In one or more implementations, the pre-training framework 400 can generalize to PPG and ECG physiological signals. In one or more other implementations, the pre-training framework 400 can incorporate modality-specific embeddings. In one or more other implementations, the pre-training framework 400 can incorporate longitudinal changes in PPG and ECG embeddings. In one or more other implementations, the pre-training framework 400 can incorporate multi-modal pre-training by either using a multi-modal encoder or a CLIP-style approach, to leverage multiple modalities simultaneously. In one or more other implementations, the pre-training framework 400 can incorporate different positive pair selection strategies, considering temporal and other physiological information to significantly influence the quality of the embeddings.

At block 308, the apparatus may optionally deploy the trained machine learning model to predict a physiological state of the user.

FIG. 5A illustrates an example encoder architecture in accordance with one or more implementations. FIG. 5A illustrates the architecture of the encoder 440 configured for processing time-series input data (e.g., input segment 510). It delineates the components of the encoder architecture, including convolutional blocks denoted as Conv1D (e.g., 522), batch normalization layers represented by BatchNorm (e.g., 524), activation functions such as Swish (e.g., 526), mobile inverted bottleneck blocks referred to as MBConv1D (e.g., 528), and average pooling operations tailored for one-dimensional data, labeled as 1DAvgPool (e.g., 530). The encoder 440 for the input segment 510 containing PPG data can include about 3.3 million parameters, while for the input segment 510 containing ECG data, the encoder 440 can consist about 2.5 million parameters.

In one or more other implementations, the encoder 440 architecture incorporates residual connections tailored for processing 1D time-series data. In one or more other implementations, the encoder 440 architecture includes a 6-layer convolutional neural network for token embedding, resulting in 60 tokens with 256 dimensions. This can be followed by an 8-layer transformer with 8 attention heads and a 1024 MLP feedforward dimension. In one or more implementations, global average pooling can be applied to the output tokens to obtain the final 256-dimensional embedding. In one or more implementations, a linear learning rate warm-up, starting from 50% of the maximum learning rate for the initial 10 epochs, is incorporated to enhance stability and optimize training performance.

FIG. 5B illustrates an example architecture of a mobile inverted bottleneck convolutional block 528 as described with reference to FIG. 5A in accordance with one or more implementations. The internal structure of the MBConv1D block 528 illustrates the usage of a Sigmoid activation function 550 and element-wise multiplication operations 552 depicted by asterisks. This architecture can extract features from time-series data, employing a combination of specialized layers and blocks optimized for effective processing and feature representation.

FIG. 6 conceptually illustrates an example overview of comparison of predictions between different types of embeddings using large-scale training of foundation models in accordance with one or more implementations. In one or more implementations, physiological signal information (including health information) of users is encoded by PPG and ECG foundation models. In some aspects, the linear classification accuracy of numerous binary targets can be assessed using embeddings and compared with baseline features, which include age, biological gender, BMI, ethnicity, average heart rate, and standard deviation of heart rate derived from PPG physiological signals. The comparison of predictions (a) using PPG embeddings (e.g., scatter plot 610), (b) using ECG embeddings (e.g., scatter plot 620), as opposed to baseline features, is depicted in FIG. 6. Each physiological state target can be represented by a marker, with the y-axis indicating the ROC AUC of binary classification utilizing the embeddings, and the x-axis indicating the same for the baseline features.

The amount of information encoded in the embeddings (the representation after the deep encoder) can be computed through linear probing. In some aspects, FIG. 6 may illustrate evaluation of the performance of linear classification or regression in predicting age, biological gender, and BMI for both PPG and ECG embeddings. It can be observed that PPG embeddings demonstrate superior predictive capabilities for these physiological state targets, indicating that PPG physiological signals may contain more information pertaining to users' physiological state or health conditions. For example, the PPG embeddings (as illustrated in scatter plot 610) may outperform baseline features in predicting health conditions, suggesting that the embeddings in the PPG foundation models can readily capture information regarding users beyond what could be predicted based on user demographics and heart rate-related data. Similarly, the ECG embeddings (as illustrated in scatter plot 620) may outperform baseline features in predicting health conditions. In comparison between the PPG and ECG embeddings, ECG embeddings may encode less information pertaining to users than PPG embeddings. In some aspects, this may be attributed to various factors, including the possibility that ECG physiological signals contain less relevant information concerning these health conditions of users.

As described above, one aspect of the present technology is the gathering and use of data available from specific and legitimate sources for predicting a physiological state of a user from physiological signals from wearable electronic devices using large-scale training of foundation models. The present disclosure contemplates that in some instances, this gathered data may include personal information data that uniquely identifies or can be used to identify a specific person. Such personal information data can include audio data, demographic data, location-based data, online identifiers, telephone numbers, email addresses, home addresses, biometric data or records relating to a user's health or level of fitness (e.g., vital signs measurements, medication information, exercise information, motion information, heartrate information workout information), date of birth, or any other personal information.

The present disclosure recognizes that the use of such personal information data, in the present technology, can be used to the benefit of users. For example, the personal information data can be used for capturing personal biometric data including physiological signals.

The present disclosure contemplates that those entities responsible for the collection, analysis, disclosure, transfer, storage, or other use of such personal information data will comply with well-established privacy policies and/or privacy practices. In particular, such entities would be expected to implement and consistently apply privacy practices that are generally recognized as meeting or exceeding industry or governmental requirements for maintaining the privacy of users. Such information regarding the use of personal data should be prominently and easily accessible by users, and should be updated as the collection and/or use of data changes. Personal information from users should be collected for legitimate uses only. Further, such collection/sharing should occur only after receiving the consent of the users or other legitimate basis specified in applicable law. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations which may serve to impose a higher standard. For instance, in the US, collection of or access to certain health data may be governed by federal and/or state laws, such as the Health Insurance Portability and Accountability Act (HIPAA); whereas health data in other countries may be subject to other regulations and policies and should be handled accordingly.

Despite the foregoing, the present disclosure also contemplates aspects in which users selectively block the use of, or access to, personal information data. That is, the present disclosure contemplates that hardware and/or software elements can be provided to prevent or block access to such personal information data. For example, in the example of large-scale training of foundation models for predicting a physiological state of a user from physiological signals on wearable electronic devices, the present technology can be configured to allow users to select to “opt in” or “opt out” of participation in the collection and/or sharing of personal information data during registration for services or anytime thereafter. In addition to providing “opt in” and “opt out” options, the present disclosure contemplates providing notifications relating to the access or use of personal information. For instance, a user may be notified upon downloading an application that their personal information data will be accessed and then reminded again just before personal information data is accessed by the application.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing identifiers, controlling the amount or specificity of data stored (e.g., collecting location data at city level rather than at an address level or at a scale that is insufficient for facial recognition), controlling how data is stored (e.g., aggregating data across users), and/or other methods such as differential privacy.

Therefore, although the present disclosure broadly covers use of personal information data to implement one or more various disclosed implementations, the present disclosure also contemplates that the various implementations can also be implemented without the need for accessing such personal information data. That is, the various implementations of the present technology are not rendered inoperable due to the lack of all or a portion of such personal information data.

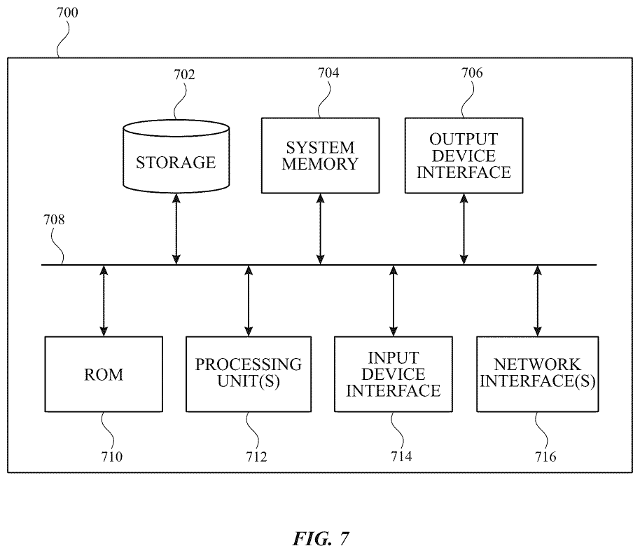

FIG. 7 illustrates an electronic system 700 with which one or more implementations of the subject technology may be implemented. The electronic system 700 can be, and/or can be a part of, the electronic device 110, and/or the server 120 shown in FIG. 1. The electronic system 700 may include various types of computer readable media and interfaces for various other types of computer readable media. The electronic system 700 includes a bus 708, one or more processing unit(s) 712, a system memory 704 (and/or buffer), a ROM 710, a permanent storage device 702, an input device interface 714, an output device interface 706, and one or more network interfaces 716, or subsets and variations thereof.

The bus 708 collectively represents all system, peripheral, and chipset buses that communicatively connect the numerous internal devices of the electronic system 700. In one or more implementations, the bus 708 communicatively connects the one or more processing unit(s) 712 with the ROM 710, the system memory 704, and the permanent storage device 702. From these various memory units, the one or more processing unit(s) 712 retrieves instructions to execute and data to process in order to execute the processes of the subject disclosure. The one or more processing unit(s) 712 can be a single processor or a multi-core processor in different implementations.

The ROM 710 stores static data and instructions that are needed by the one or more processing unit(s) 712 and other modules of the electronic system 700. The permanent storage device 702, on the other hand, may be a read-and-write memory device. The permanent storage device 702 may be a non-volatile memory unit that stores instructions and data even when the electronic system 700 is off. In one or more implementations, a mass-storage device (such as a magnetic or optical disk and its corresponding disk drive) may be used as the permanent storage device 702.

In one or more implementations, a removable storage device (such as a flash drive, and its corresponding solid state drive) may be used as the permanent storage device 702. Like the permanent storage device 702, the system memory 704 may be a read-and-write memory device. However, unlike the permanent storage device 702, the system memory 704 may be a volatile read-and-write memory, such as random access memory. The system memory 704 may store any of the instructions and data that one or more processing unit(s) 712 may need at runtime. In one or more implementations, the processes of the subject disclosure are stored in the system memory 704, the permanent storage device 702, and/or the ROM 710. From these various memory units, the one or more processing unit(s) 712 retrieves instructions to execute and data to process in order to execute the processes of one or more implementations.

The bus 708 also connects to the input device interface 714 and output device interface 706. The input device interface 714 enables a user to communicate information and select commands to the electronic system 700. Input devices that may be used with the input device interface 714 may include, for example, alphanumeric keyboards and pointing devices (also called “cursor control devices”). The output device interface 706 may enable, for example, the display of images generated by electronic system 700. Output devices that may be used with the output device interface 706 may include, for example, printers and display devices, such as a liquid crystal display (LCD), a light emitting diode (LED) display, an organic light emitting diode (OLED) display, a flexible display, a flat panel display, a solid state display, a projector, or any other device for outputting information. One or more implementations may include devices that function as both input and output devices, such as a touchscreen. In these implementations, feedback provided to the user can be any form of sensory feedback, such as visual feedback, auditory feedback, or tactile feedback; and input from the user can be received in any form, including acoustic, speech, or tactile input.

Finally, as shown in FIG. 7, the bus 708 also couples the electronic system 700 to one or more networks and/or to one or more network nodes, such as the electronic device 110 shown in FIG. 1, through the one or more network interface(s) 716. In this manner, the electronic system 700 can be a part of a network of computers (such as a LAN, a wide area network (“WAN”), or an Intranet, or a network of networks, such as the Internet. Any or all components of the electronic system 700 can be used in conjunction with the subject disclosure.

Implementations within the scope of the present disclosure can be partially or entirely realized using a tangible computer-readable storage medium (or multiple tangible computer-readable storage media of one or more types) encoding one or more instructions. The tangible computer-readable storage medium also can be non-transitory in nature.

The computer-readable storage medium can be any storage medium that can be read, written, or otherwise accessed by a general purpose or special purpose computing device, including any processing electronics and/or processing circuitry capable of executing instructions. For example, without limitation, the computer-readable medium can include any volatile semiconductor memory, such as RAM, DRAM, SRAM, T-RAM, Z-RAM, and TTRAM. The computer-readable medium also can include any non-volatile semiconductor memory, such as ROM, PROM, EPROM, EEPROM, NVRAM, flash, nvSRAM, FeRAM, FeTRAM, MRAM, PRAM, CBRAM, SONOS, RRAM, NRAM, racetrack memory, FJG, and Millipede memory.

Further, the computer-readable storage medium can include any non-semiconductor memory, such as optical disk storage, magnetic disk storage, magnetic tape, other magnetic storage devices, or any other medium capable of storing one or more instructions. In one or more implementations, the tangible computer-readable storage medium can be directly coupled to a computing device, while in other implementations, the tangible computer-readable storage medium can be indirectly coupled to a computing device, e.g., via one or more wired connections, one or more wireless connections, or any combination thereof.

Instructions can be directly executable or can be used to develop executable instructions. For example, instructions can be realized as executable or non-executable machine code or as instructions in a high-level language that can be compiled to produce executable or non-executable machine code. Further, instructions also can be realized as or can include data. Computer-executable instructions also can be organized in any format, including routines, subroutines, programs, data structures, objects, modules, applications, applets, functions, etc. As recognized by those of skill in the art, details including, but not limited to, the number, structure, sequence, and organization of instructions can vary significantly without varying the underlying logic, function, processing, and output.

While the above discussion primarily refers to microprocessor or multi-core processors that execute software, one or more implementations are performed by one or more integrated circuits, such as ASICs or FPGAs. In one or more implementations, such integrated circuits execute instructions that are stored on the circuit itself.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. Skilled artisans may implement the described functionality in varying ways for each particular application. Various components and blocks may be arranged differently (e.g., arranged in a different order, or partitioned in a different way) all without departing from the scope of the subject technology.

It is understood that any specific order or hierarchy of blocks in the processes disclosed is an illustration of example approaches. Based upon design preferences, it is understood that the specific order or hierarchy of blocks in the processes may be rearranged, or that all illustrated blocks be performed. Any of the blocks may be performed simultaneously. In one or more implementations, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

As used in this specification and any claims of this application, the terms “base station”, “receiver”, “computer”, “server”, “processor”, and “memory” all refer to electronic or other technological devices. These terms exclude people or groups of people. For the purposes of the specification, the terms “display” or “displaying” means displaying on an electronic device.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The predicate words “configured to”, “operable to”, and “programmed to” do not imply any particular tangible or intangible modification of a subject, but, rather, are intended to be used interchangeably. In one or more implementations, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

Phrases such as an aspect, the aspect, another aspect, some aspects, one or more aspects, an implementation, the implementation, another implementation, some implementations, one or more implementations, an embodiment, the embodiment, another embodiment, some implementations, one or more implementations, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

The word “exemplary” is used herein to mean “serving as an example, instance, or illustration”. Any embodiment described herein as “exemplary” or as an “example” is not necessarily to be construed as preferred or advantageous over other implementations. Furthermore, to the extent that the term “include”, “have”, or the like is used in the description or the claims, such term is intended to be inclusive in a manner similar to the term “comprise” as “comprise” is interpreted when employed as a transitional word in a claim.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for”.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but are to be accorded the full scope consistent with the language claims, wherein reference to an element in the singular is not intended to mean “one and only one” unless specifically so stated, but rather “one or more”. Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the subject disclosure.

What is claimed is:

1. A method, comprising:
receiving input data comprising physiological signal information associated with a user;
generating an augmented version of the input data; and
training a neural network to attract representations of positive pairs and repel representations of negative pairs using the augmented version of the input data to produce a trained machine learning model to predict a physiological state of the user.

2. The method of claim 1, wherein generating the augmented version of the input data comprises:
assigning one or more probability values to a set of time-series augmentation functions; and
applying the set of time-series augmentation functions with the one or more probability values to the input data.

3. The method of claim 1, wherein the neural network is trained to produce embeddings that distinguish between positive pairs and negative pairs.

4. The method of claim 1, wherein training the neural network comprises encoding the augmented version of the input data into an embedding in latent space having a first number of dimensions.

5. The method of claim 4, further comprising mapping the embedding into a representation in representation space having a second number of dimensions smaller than the first number of dimensions.

6. The method of claim 1, further comprising deploying the trained machine learning model using learned representations of the physiological signal information to predict a physiological state of the user.

7. The method of claim 1, wherein the neural network is trained using self-supervised contrastive learning.

8. The method of claim 1, wherein the physiological signal information comprises one or more of photoplethysmography (PPG) data or electrocardiogram (ECG) data.

9. A device, comprising:
a memory; and
one or more processors configured to:
receive input data comprising a plurality of physiological signal information segments associated with a user;
apply one or more augmentation functions to the plurality of physiological signal information segments associated with the user to generate an augmented version of the plurality of physiological signal information segments;
train a neural network to produce a trained machine learning model by:
generating, via an encoder, an embedding of the augmented version of the plurality of physiological signal information segments, the embedding having a first number of dimensions in an embedding space;
mapping, via a multilayer perceptron projection, the embedding into a representation having a second number of dimensions smaller than the first number of dimensions; and
determining mutual information between a pair of representations of the augmented version of the plurality of physiological signal information segments associated with the user; and
deploy the trained machine learning model to predict a physiological state of the user.

10. The device of claim 9, wherein the one or more processors are further configured to assign one or more probability values to the one or more augmentation functions, wherein the one or more augmentation functions are applied to the plurality of physiological signal information segments with the one or more probability values.

11. The device of claim 9, wherein the neural network is trained to produce latent representations that distinguish between positive pairs and negative pairs.

12. The device of claim 9, wherein the neural network is trained to bring representations of positive pairs closer together in the embedding space while pushing apart representations of negative pairs.

13. The device of claim 9, wherein the mutual information is determined using a contrastive loss function.

14. The device of claim 9, wherein the neural network is trained using self-supervised contrastive learning.

15. The device of claim 9, wherein the plurality of physiological signal information segments comprises one or more of photoplethysmography (PPG) data or electrocardiogram (ECG) data.

16. A non-transitory machine-readable medium comprising code that, when executed by a processor, causes the processor to perform operations comprising:
receiving input data comprising physiological signal information associated with a user;
assigning one or more probability values to a set of time-series augmentation functions;
applying the set of time-series augmentation functions with the one or more probability values to the input data to generate an augmented version of the input data; and
training a neural network to attract representations of positive pairs and repel representations of negative pairs using the augmented version of the input data to produce a trained machine learning model to predict a physiological state of the user.

17. The non-transitory machine-readable medium of claim 16, wherein the neural network is trained to produce embeddings that distinguish between positive pairs and negative pairs.

18. The non-transitory machine-readable medium of claim 16, wherein training the neural network comprises encoding the augmented version of the input data into an embedding in latent space having a first number of dimensions.

19. The non-transitory machine-readable medium of claim 18, wherein training the neural network further comprises mapping the embedding into a representation in representation space having a second number of dimensions smaller than the first number of dimensions.

20. The non-transitory machine-readable medium of claim 16, wherein the neural network is trained using self-supervised contrastive learning.
